(19) United States
(12) Patent Application Publication
(10) Pub. No.: US 2019/0130598 A1
(43) Pub. Date: May 2, 2019

(54) MEDICAL APPARATUS

(71) Applicant: General Electric Company, Schenectady, NY (US)

(72) Inventors: Shigeru Chikamatsu, Tokyo (JP); Isamu Igarashi, Tokyo (JP)

(21) Appl. No.: 16/176,265

(22) Filed: Oct. 31, 2018

(30) Foreign Application Priority Data
Oct. 31, 2017 (JP) 2017-211002

(51) Int. Cl. G06T 7/70; G06T 7/521; G06T 7/20; A61B 5/055; A61B 6/04

(52) U.S. Cl. CPC G06T 7/70; G06T 7/521; G06T 7/20; G06T 2207/10028; A61B 6/0428; G06T 2207/10024; A61B 5/0555

(57) ABSTRACT

A CT apparatus comprises: a gantry; a table on which a subject to be examined is laid; a sensor section for acquiring distance data for determining a distance between a body part to be imaged of the subject and the sensor section; a height-data generating section for generating height data containing data of a height of the body part to be imaged of the subject based on the distance data; and a detecting section for detecting said body part to be imaged based on the height data, wherein the sensor section has a field-of-view region RV representing a region in which it is possible to acquire the distance data, and the field-of-view region RV is defined so that a portion of the table on a side of the gantry falls within the field-of-view region RV and a portion of the table on a side opposite to the side of the gantry falls outside the field-of-view region RV.

### TECHNICAL FIELD

The present invention relates to a medical apparatus for detecting a body part to be imaged of a subject to be examined, and a program applied to the medical apparatus.

### BACKGROUND

Medical apparatuses, such as a CT (Computed Tomography) apparatus and an MRI (Magnetic Resonance Imaging) apparatus, are known as apparatuses for acquiring an image of the inside of a subject to be examined Since the CT and MRI apparatuses are capable of non-invasively imaging the subject, they are used as apparatuses indispensable in diagnosing the subject's health.

On the other hand, in imaging the subject with the CT apparatus and MRI apparatus, a radiographer has to carry out various tasks for preparing a scan, which poses a problem that radiographers experience a lot of work stress. Accordingly, to mitigate the radiographers' work stress, there is disclosed a technique of automatically operating a table of a CT apparatus (see PTL 1).

According to Japanese Patent Application Publication No. 2014-161392, an oblique bird's-eye view image is produced based on data obtained from a camera, and a direct bird's-eye view image is produced from the oblique one based on data obtained from a depth sensor. The table is then moved based on the direct bird's-eye view image; such an attempt is made therein.

In imaging the subject, the body part to be imaged of the subject should be positioned within a bore of a gantry. Therefore, it is important in the technique of automatic control of the table using the camera and depth sensor to detect the body part to be imaged of the subject from the direct bird's-eye view image, and control the table so that the detected body part to be imaged is positioned within the bore of the gantry. When the camera and depth sensor are configured so that the whole table falls within a field-of-view region of the camera and depth sensor as in PTL 1, however, resolution of an image of the body part to be imaged lowers and/or image distortion is more likely to occur as the body part to be imaged lies farther away from the camera and depth sensor, which poses a problem that poor accuracy of detection of the body part to be imaged results.

Therefore, there is a need for a technique with which it is possible to improve accuracy of detection of a body part to be imaged.

### SUMMARY

A first aspect of the present invention is a medical apparatus comprising:

a gantry;

a table on which a subject to be examined is laid;

a sensor section for acquiring distance data for determining a distance between a body part to be imaged of said subject and said sensor section;

height-data generating means for generating height data containing data representing a height of the body part to be imaged of said subject based on said distance data; and

detecting means for detecting said body part to be imaged based on said height data, wherein

said sensor section has a field-of-view region representing a region in which it is possible to acquire said distance data, and

said field-of-view region is defined so that a portion of said table on a side of said gantry falls within said field-of-view region and a portion of said table on a side opposite to the side of said gantry falls outside said field-of-view region.

A second aspect of the present invention is a program applied to a medical apparatus comprising: a gantry; a table on which a subject to be examined is laid; and a sensor section for acquiring distance data for determining a distance between a body part to be imaged of said subject and said sensor section, wherein said sensor section has a field-of-view region representing a region in which it is possible to acquire said distance data, and said field-of-view region is defined so that a portion of said table on a side of said gantry falls within said field-of-view region and a portion of said table on a side opposite to the side of said gantry falls outside said field-of-view region, said program causing a computer to execute:

height-data generating processing of generating height data containing data representing a height of the body part to be imaged of said subject based on said distance data; and

detecting processing of detecting said body part to be imaged based on said height data.

The portion of the table on the side of the gantry falls within the field-of-view region of the sensor section, while the portion of the table on the side opposite to the gantry falls outside the field-of-view region. Thus, the sensor section can be installed closer to the subject, so that accuracy of detection of distance data from the sensor section can be improved, and as a result, accuracy of detection of the body part to be imaged can be improved.

### BRIEF DESCRIPTION OF DRAWINGS

FIG. 1 is an external view of an X-ray CT apparatus in an embodiment of the present invention;

FIG. 2 is a diagram schematically showing a hardware configuration of the X-ray CT apparatus 1 in accordance with the present embodiment;

FIG. 3 is an explanatory diagram for a sensor section 19 and a display section 18;

FIG. 4 is a diagram showing a case in which a portion 4b of a table 4 on a side opposite to the gantry 2 falls outside a field-of-view region RV of the sensor section 19;

FIG. 5 is a diagram showing a case in which the entire table 4 falls within a field-of-view region RV′ of the sensor section 19;

FIG. 6 is a block diagram of main functions of the X-ray CT apparatus;

FIG. 7 is a diagram showing an exemplary operation flow in the present embodiment;

FIG. 8 is a diagram showing a condition in which the subject 5 is laid on a cradle 41 of the table 4;

FIG. 9 is a diagram showing a condition in which the cradle 41 moves in a y-direction by Δy=Δy1;

FIG. 10 is a diagram showing a condition in which the cradle 41 has reached y=y2;

FIG. 11 is a diagram showing a condition in which the cradle 41 moves from z=z0 to z=z1;

FIG. 12 is a diagram showing a condition in which the cradle 41 has reached z=z2;

FIG. 13 is a diagram showing a condition in which the cradle 41 has moved by Δy_{c} and Δz_{c}; and

FIG. 14 is a diagram showing a condition in which a body part to be imaged has reached a center position zⱼ of the field-of-view region RV in a z-direction.

### DETAILED DESCRIPTION

Now an embodiment for practicing the invention will be described hereinbelow, although the present invention is not limited to the following embodiment.

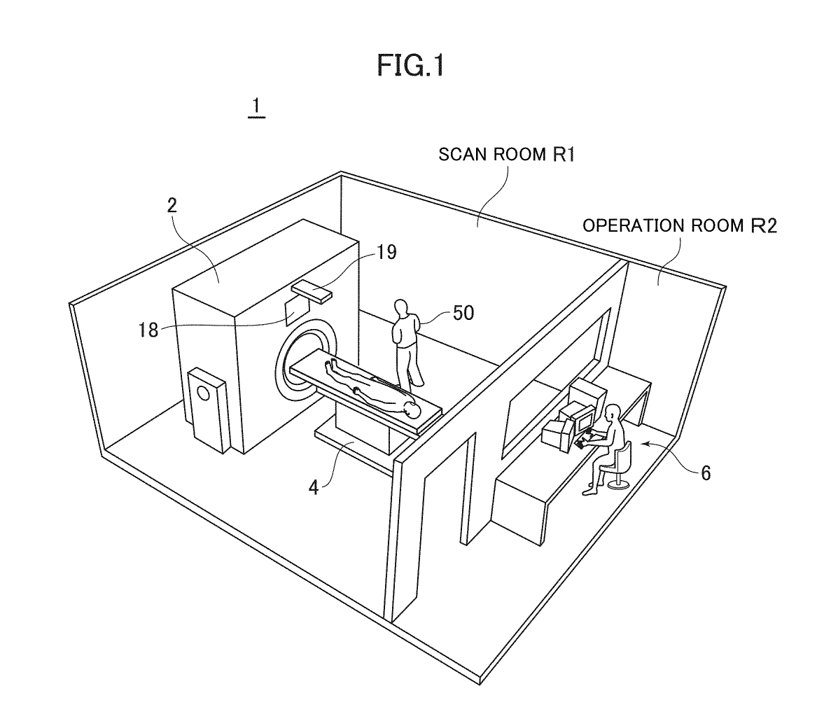

FIG. 1 is an external view of an X-ray CT apparatus in the present embodiment.

As shown in FIG. 1, the X-ray CT apparatus 1 comprises a gantry 2, a table 4, and an operation console 6.

The gantry 2 and table 4 are installed in a scan room RE The operation console 6 is installed in an operation room R2 different from the scan room R1.

The gantry 2 is provided on its front surface with a sensor section 19 and a display section 18. The sensor section 19 and display section 18 will be discussed later.

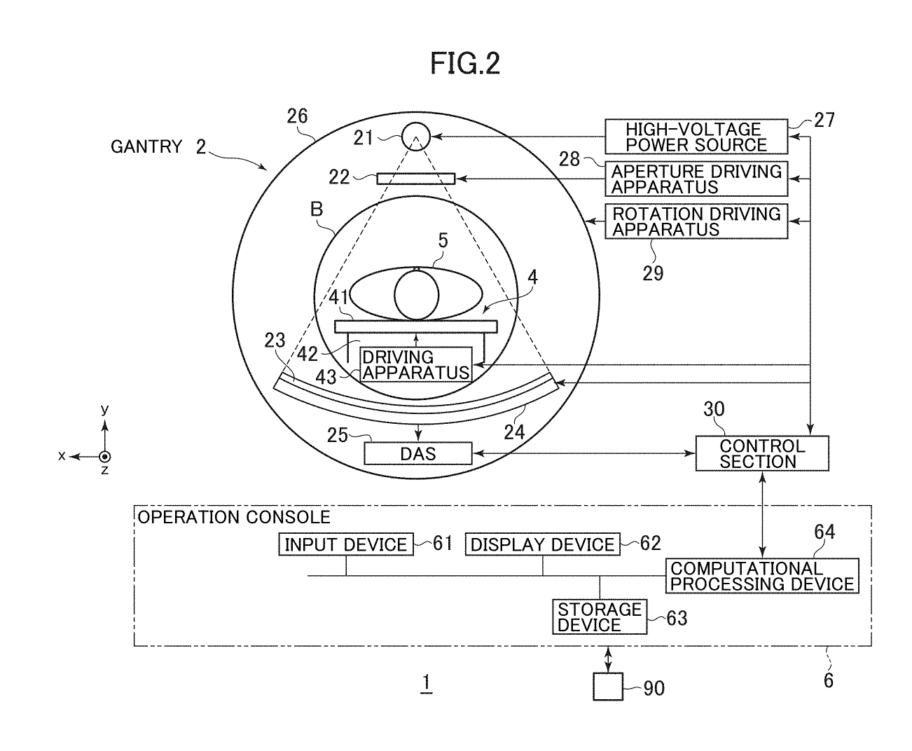

FIG. 2 is a diagram schematically showing a hardware configuration of the X-ray CT apparatus 1 in accordance with the present embodiment.

As used herein, a direction corresponding to that of the body axis of the subject 5 will be referred to as z-direction, as shown in FIG. 2. A direction corresponding to a vertical direction (direction of gravity) will be referred to as y-direction, and a direction orthogonal to the y- and z-directions will be referred to as x-direction.

The gantry 2 has an X-ray tube 21, an aperture 22, a collimator device 23, an X-ray detector 24, a data acquisition system (DAS) 25, a rotating section 26, a high-voltage power source 27, an aperture drive apparatus 28, a rotation drive apparatus 29, and a control section 30. In FIG. 2, the sensor section 19 and display section 18 provided on the front surface of the gantry 2 are omitted in the drawing.

The X-ray tube 21, aperture 22, collimator device 23, X-ray detector 24, and data acquisition system 25 are mounted on the rotating section 26.

The X-ray tube 21 and X-ray detector 24 are disposed to face each other sandwiching an imaging volume, i.e., a bore B of the gantry 2, in which a subject 5 to be examined is placed.

The aperture 22 is disposed between the X-ray tube 21 and bore B. The aperture 22 shapes X-rays emitted from an X-ray focus of the X-ray tube 21 toward the X-ray detector 24 into a fan beam or a cone beam.

The collimator device 23 is disposed between the bore B and X-ray detector 24. The collimator device 23 removes scatter rays that would otherwise impinge upon the X-ray detector 24.

The X-ray detector 24 has a plurality of X-ray detector elements two-dimensionally arranged in directions of the span and the thickness of the fan-shaped X-ray beam emitted from the X-ray tube 21. The X-ray detector elements each detect X-rays passing through the subject 5 placed in the bore B, and output electric signals depending upon their intensity.

The data acquisition system 25 receives the electric signals output from the X-ray detector elements in the X-ray detector 24, and converts them into X-ray data for collection.

The table 4 has a cradle 41, a support base 42, and a drive apparatus 43. The subject 5 is laid on the cradle 41. The support base 42 supports the cradle 41. The drive apparatus 43 drives the cradle 41 and support base 42 so that the cradle 41 is moved in the y- and z-directions.

The high-voltage power source 27 supplies high voltage and electric current to the X-ray tube 21.

The aperture drive apparatus 28 drives the aperture 22 to modify the shape of its opening.

The rotation drive apparatus 29 rotationally drives the rotating section 26.

The control section 30 controls several apparatuses and sections in the gantry 2, the drive apparatus 43 of the table 4, etc.

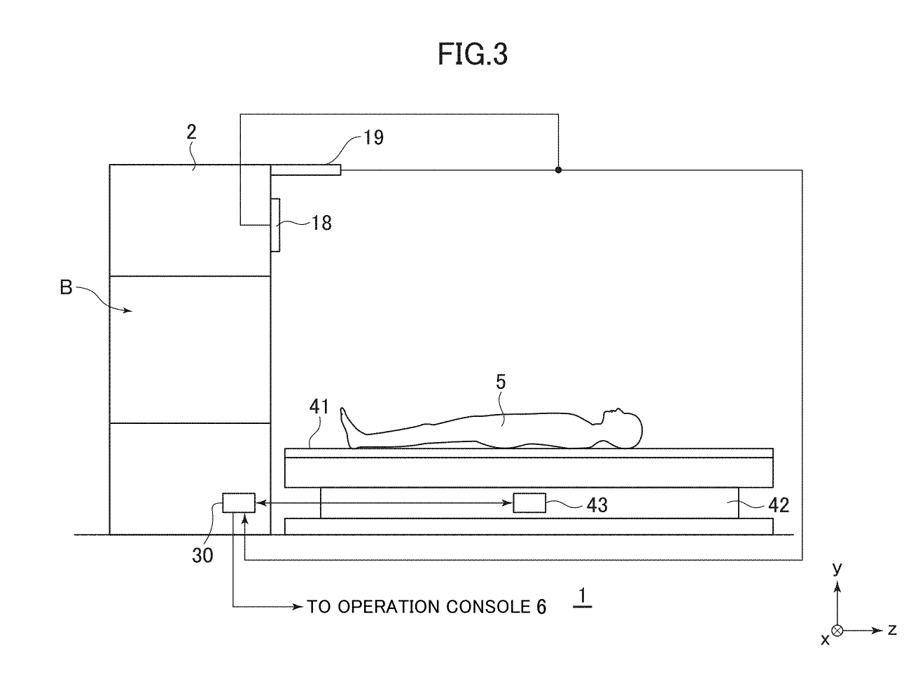

FIG. 3 is an explanatory diagram for the sensor section 19 and display section 18. FIG. 3 shows a side view of the gantry 2 and table 4.

The display section 18 has a display with touch-panel-driven GUI (Graphical User Interface). The display section 18 is connected to the operation console 6 via the control section 30. A radiographer performs a touch-panel operation on the display section 18, whereby he/she can achieve several kinds of operations and settings related to the X-ray CT apparatus 1. The display section 18 can also display several kinds of setting screens, graph displays, images, etc., on its display.

The sensor section 19 has a number of pixels of n by m, and is configured to acquire image data and distance data. The numbers n and m are, for example, n=640 and m=480. Each pixel in the sensor section 19 has an imaging section for acquiring the image data.

The imaging section is a CCD (Charge Coupled Device) for acquiring color information in, for example, RGB (Red Green Blue), or a monochrome CCD. The image data for the subject 5 can be acquired by the imaging section.

Moreover, in addition to the imaging section, each pixel in the sensor section 19 is provided with a light-receiving section for acquiring the distance data. The light-receiving section receives reflected light of light emitted from a light source (not shown) provided in the sensor section 19 onto the subject 5. Based on the received reflected light, the sensor section 19 outputs the distance data for determining a distance from the sensor section 19 to each position on the surface of the subject 5. The sensor section 19 that may be used is, for example, a TOF camera manufactured by Panasonic Photo & Lighting Co., Ltd. The light source that may be used is, for example, an infrared source, a laser light source, or the like.

The control section 30 drives the drive apparatus 43 as needed based on input signals from the display section 18 and/or sensor section 19.

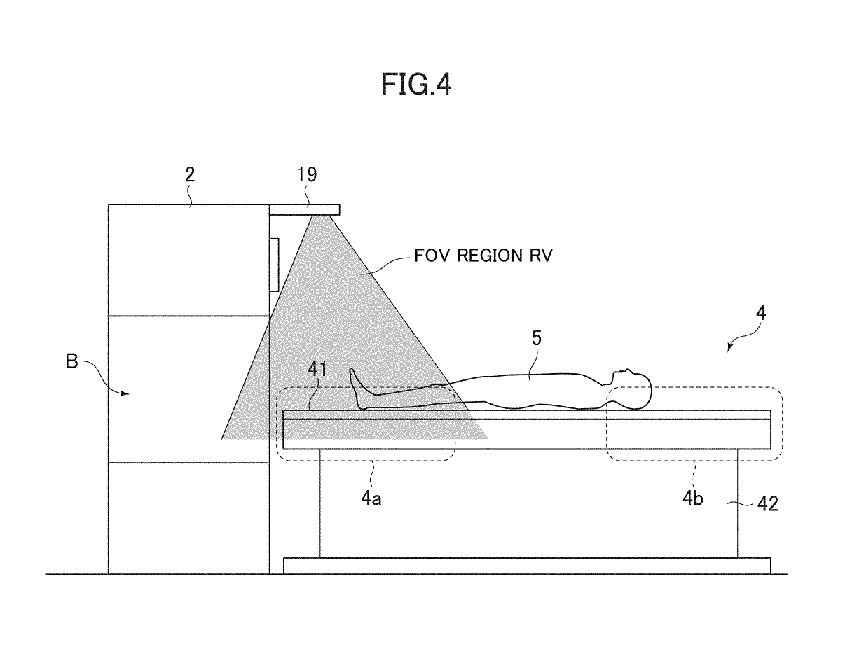

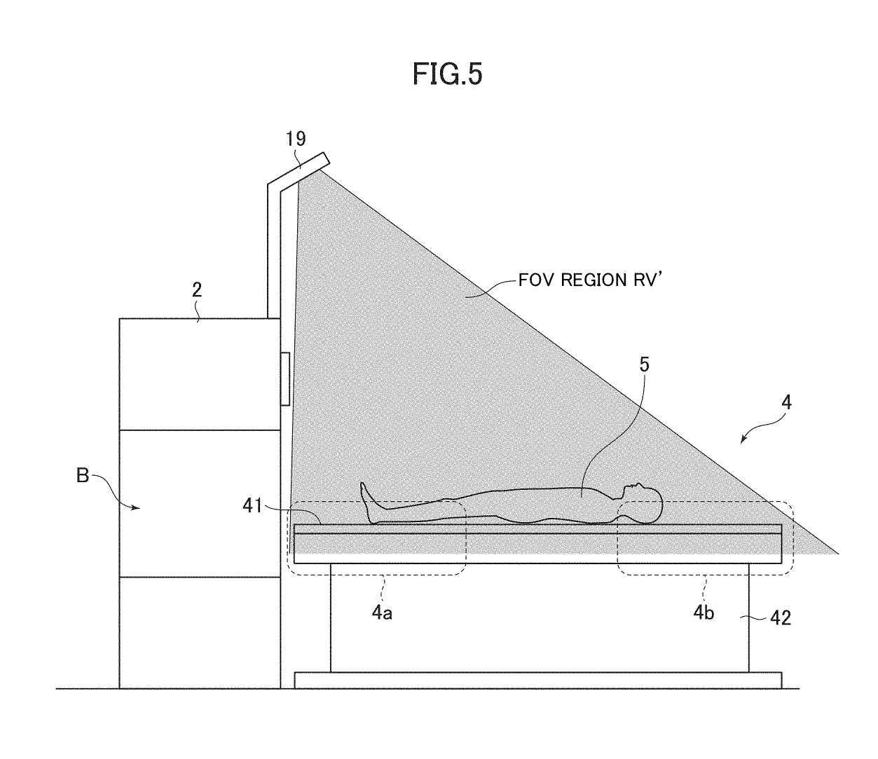

FIGS. 4 and 5 are explanatory diagrams for a field-of-view (FOV) region of the sensor section 19.

FIGS. 4 and 5 show two field-of-view regions.

Now FIG. 4 will be described first.

In FIG. 4, a field-of-view region of the sensor section 19 is defined so that a portion 4a of the table 4 on the side of the gantry 2 falls within the field-of-view region RV while a portion 4b of the table 4 on the side opposite to the gantry 2 falls outside the field-of-view region RV. In FIG. 4, the field-of-view region RV represents a region in which the sensor section is capable of acquiring distance data and image data.

On the other hand, FIG. 5 shows a case in which the orientation and position of the sensor section 19 are defined so that the whole table 4 falls within a field-of-view region RV of the sensor section 19. Thus, in FIG. 5, there is an advantage that an image of the whole body of the subject 5 can be acquired.

In the case of FIG. 5, however, the sensor section 19 should be installed so that the total length of the table 4 falls within the field-of-view region RV′, and thus, the sensor section 19 in FIG. 5 is installed at a position farther away from the subject 5 than the sensor section 19 is in FIG. 4. This poses a problem that accuracy of detection of the distance data is poorer for the sensor section 19 in FIG. 5 than that in FIG. 4. In particular, the portion 4b of the table 4 away from the gantry 2 is farther from the sensor section 19 than the portion 4a of the table 4 on the side of the gantry 2 is. Therefore, there is a problem that accuracy of detection of the distance data is likely to be poorer in the portion 4b of the table 4 farther away from the gantry 2.

There is also another problem that the sensor section 19 in FIG. 5 provides poorer image resolution and aggravated image distortion in the portion 4b of the table 4 farther away from the gantry 2.

The sensor section in FIG. 4, on the other hand, has the portion 4b of the table 4 on the side opposite to the gantry 2 falling outside the field-of-view region RV, and therefore, it can be installed at a position closer to the subject 5 than the sensor section 19 in FIG. 5 can. Therefore, accuracy of detection of the distance data can be improved for the sensor section in FIG. 4 relative to the sensor section 19 in FIG. 5. Moreover, since the sensor section in FIG. 4 has a smaller field-of-view region than that of the sensor section 19 in FIG. 5, a region to be detected by the imaging section can be smaller, thus enhancing image resolution or reducing image distortion.

Accordingly, in the present embodiment, the field-of-view region RV of the sensor section 19 is defined so that the portion 4a of the table 4 on the side of the gantry 2 falls within the field-of-view region RV while the portion 4b of the table 4 on the side opposite to the gantry 2 falls outside the field-of-view region RV, as shown in FIG. 4.

Returning to FIG. 2, the explanation will be continued below.

The operation console 6 accepts several kinds of operations from the radiographer. The operation console 6 has an input device 61, a display device 62, a storage device 63, and a computational processing device 64.

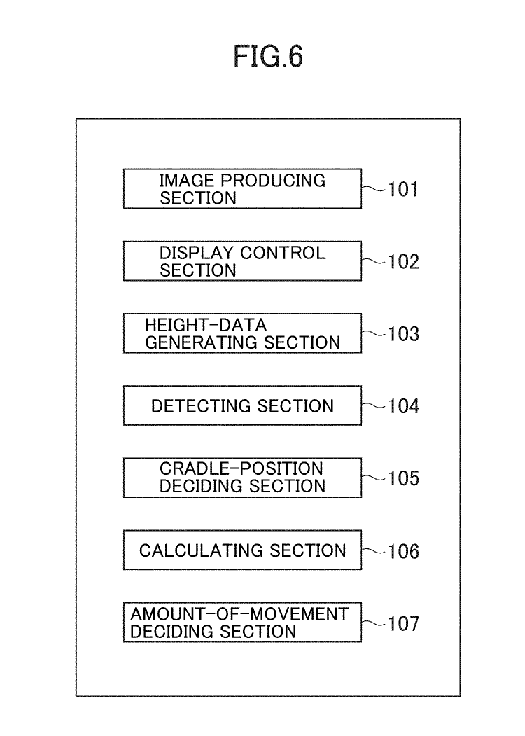

FIG. 6 is a block diagram of main functions of the X-ray CT apparatus. While in practice, the X-ray CT apparatus has a large number of functional blocks, only those necessary in the explanation of the present embodiment are shown here.

In the present embodiment, the X-ray CT apparatus has, as its main functional blocks, an image producing section 101, a display control section 102, a height-data generating section 103, a detecting section 104, a cradle-position deciding section 105, a calculating section 106, and an amount-of-movement deciding section 107.

The image producing section 101 produces an image of the subject based on image data obtained from the sensor section 19.

The display control section 102 controls the display section 18 so that the image of the subject is displayed in the display section 18.

The height-data generating section 103 generates height data containing data representing the height of the body part to be imaged of the subject based on distance data obtained by the sensor section 19.

The detecting section 104 detects the body part to be imaged of the subject 5 based on the height data.

The cradle-position deciding section 105 decides whether or not the cradle 41 has reached a prespecified position y2 in the y-direction.

The calculating section 106 calculates amounts of movement of the cradle 41 required for carrying the body part to be imaged of the subject 5 into the bore B of the gantry 2, wherein the amounts of movement of the cradle 41 are an amount Δyc of movement in the y-direction and an amount Δzc of movement in the z-direction.

The amount-of-movement deciding section 107 decides whether or not the cradle 41 has moved by the amounts Δyc and Δzc of movement.

The height-data generating section 103 constitutes an example of the height-data generating means, the detecting section 104 constitutes an example of the detecting means, and the calculating section 106 constitutes an example of the calculating means.

Programs for implementing these functional blocks may be stored in at least one of the storage section 63 in the operation console 6, a storage section in the gantry 2, and that in the table 4. At least one of the gantry 2, table 4, and operation console 6 comprises a section that serves as a computer for executing the programs stored in the storage section, and the computer functions as respective functional blocks by executing the programs stored in the storage section. It is also possible to store at least part of the programs into a storage section or a storage medium 90 (see FIG. 2) externally connected with the operation console 6. Details of the functions shown in FIG. 6 will be described later in explaining the processing flow in the X-ray CT apparatus.

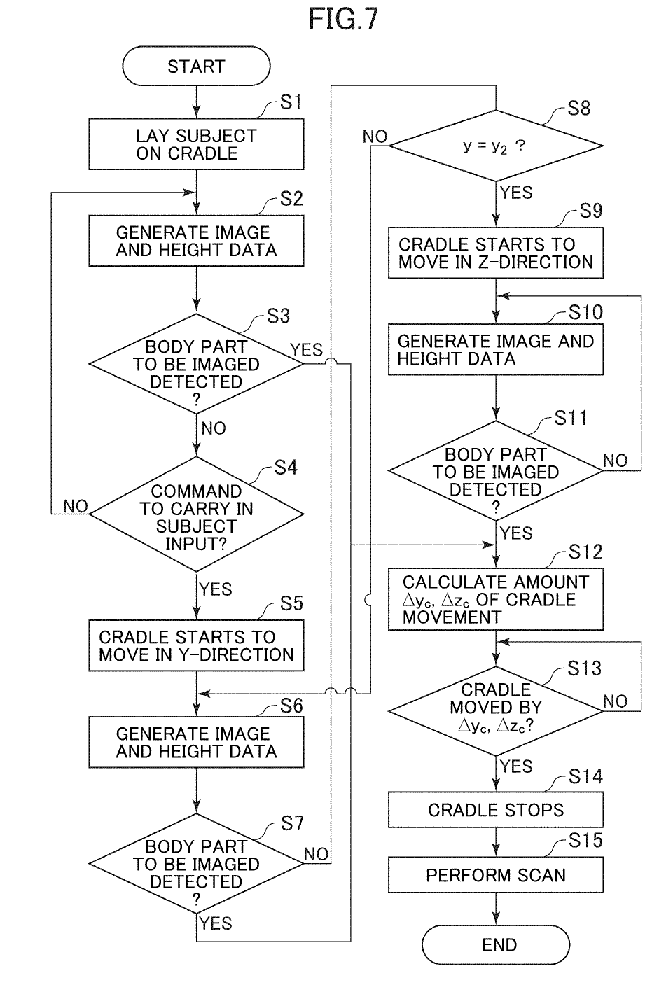

FIG. 7 is a diagram showing an exemplary operation flow in the present embodiment.

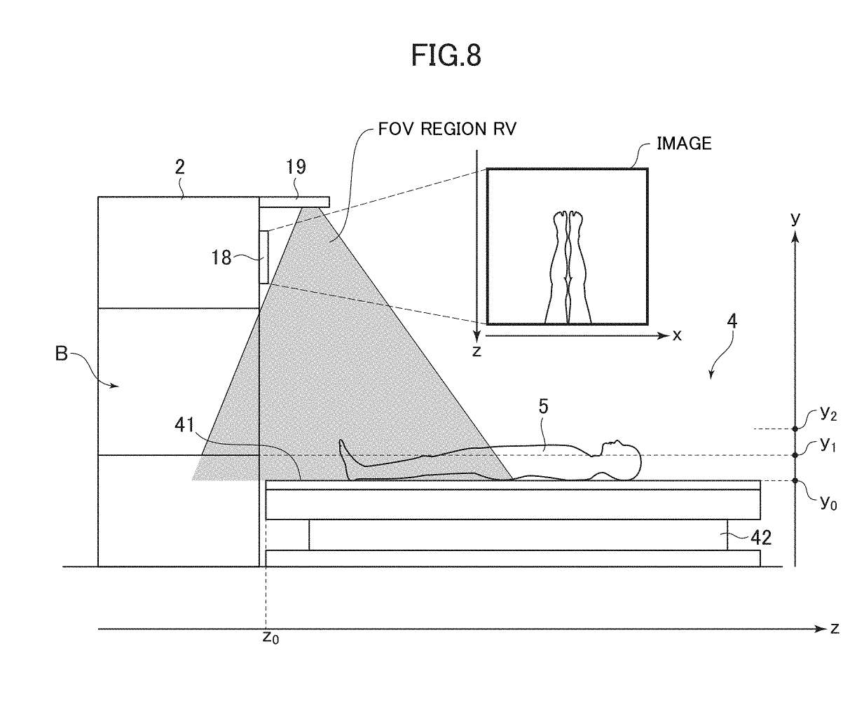

At Step S1, the radiographer lays the subject 5 on the cradle 41 of the table 4 (see FIG. 8). FIG. 8 is a diagram showing a condition in which the subject 5 is laid on the cradle 41 of the table 4. In FIG. 8, the position of the cradle 41 in the y-direction is designated by y=y0, and that in the z-direction is designated by z=z0.

The radiographer also sets scan conditions (a body part to be imaged, for example) for the subject 5. The body part to be imaged is considered here as a chest part. After the subject 5 is laid on the cradle 41 as shown in FIG. 8, the process goes to Step S2.

At Step S2, the image producing section 101 (see FIG. 6) produces an image of the subject 5 based on image data obtained from the sensor section 19. The display control section 102 (see FIG. 6) controls the display section 18 so that the image produced by the image producing section 101 is displayed in the display section 18. FIG. 8 schematically shows the image displayed in the display section 18. In FIG. 8, the leg part of the subject 5 falls within the field-of-view region RV, while the upper half body of the subject does not fall within the field-of-view region RV. Therefore, the leg part of the subject 5 is displayed in the display section 18, while the upper half body is not displayed therein. By viewing the display section 18, the radiographer can confirm what body part of the subject has entered the field-of-view region RV of the sensor section 19.

Moreover, the height-data generating section 103 (see FIG. 6) generates height data containing data representing the height of the subject to be imaged in the y-direction falling within the field-of-view region RV based on distance data obtained from the sensor section 19. Since the leg part of the subject 5 falls within the field-of-view region RV in FIG. 8, the height-data generating section 103 generates height data containing the data representing the height of the leg part of the subject. Specifically, the height data contains data representing the height in the y-direction of points on the body surface of the leg part of the subject in the zx-plane. Therefore, by obtaining the height data, a three-dimensional shape of the surface of the leg part of the subject can be known.

At Step S3, the detecting section 104 (see FIG. 6) executes processing for detecting the body part to be imaged of the subject from among the height data.

To execute the detecting processing, the detecting section 104 identifies which body part of the subject 5 is the body part to be imaged before executing the detecting processing. The detecting section 104 can identify the body part to be imaged of the subject 5 based on, for example, information that the radiographer has input from the operation console 6 and/or information that the radiographer has input from the display section 18 in the gantry 2. For example, in the case that the radiographer 50 has selected a protocol for imaging the chest part from the operation console 6, the detecting section 104 decides that the body part to be imaged of the subject 5 is the chest part. The detecting section 104 can thus identify the body part to be imaged of the subject 5.

Since the chest part is set for the body part to be imaged here, the detecting section 104 executes processing for detecting the chest part of the subject from among the height data. In the case that the body part to be imaged is detected from among the height data, the process goes to Step S12. On the other hand, in the case that the body part to be imaged is not contained in the height data, the detecting section 104 decides that the body part to be imaged cannot be detected. In this case, the process goes to Step S4. An exemplary method of detecting the body part to be imaged comprises preparing beforehand templates reflecting standard heights of positions on the body surface of body parts to be imaged, such as a head part, a shoulder part, a chest part, an abdominal part, and a leg part, and performing matching of the height data with each template while scaling up/down and rotating the height data or template, to detect the body part to be imaged. In FIG. 8, the body part to be imaged (chest part) does not fall within the field-of-view region RV. Therefore, the body part to be imaged is not detected, and the process goes to Step S4.

At Step S4, whether or not the radiographer has input a command to carry the subject 5 into the bore B of the gantry 2 is decided. In the case that the command of carrying is input, the process goes to Step S5. On the other hand, in the case that the command of carrying is not input, the process goes back to Step S2. Thus, a loop of Steps S2, S3, and S4 is repetitively executed until the command of carrying is input.

Once the radiographer has input the command to carry the subject 5 into the bore B of the gantry 2 via the input device, the process goes to Step S5.

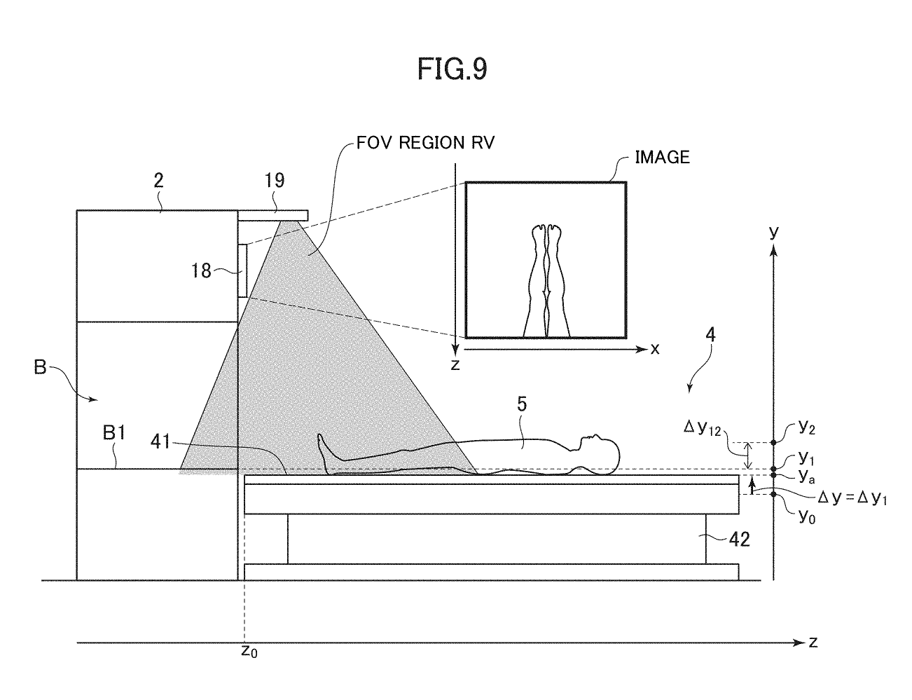

At Step S5, the control section 30 controls the table 4 so that the cradle starts moving in the y-direction. Thus, the cradle 41 starts moving in the y-direction. FIG. 9 shows a condition in which the cradle 41 has moved by Δy=Δy1 in the y-direction.

At Step S6, the image producing section 101 produces an image of the subject 5 based on image data obtained from the sensor section 19. The display control section 102 controls the display section 18 so that the image produced by the image producing section 101 is displayed in the display section 18. In FIG. 9 is schematically shown the image at a time point when the cradle 41 has moved by Δy=Δy1 in the y-direction.

Moreover, the height-data generating section 103 generates height data at the time point when the cradle 41 has moved by Δy=Δy1 in the y-direction based on distance data obtained from the sensor section 19.

At Step S7, the detecting section 104 executes detection processing for detecting the body part to be imaged of the subject from among the height data generated at Step S6. In the case that the body part to be imaged is detected, the process goes to Step S12. On the other hand, in the case that the body part to be imaged is not detected, the process goes to Step S8. In FIG. 9, the body part to be imaged (chest part) does not fall within the field-of-view region RV. Therefore, the body part to be imaged is not detected, and the process goes to Step S8.

At Step S8, the cradle-position deciding section 105 (see FIG. 6) decides whether or not the cradle 41 has reached the prespecified position y2 in the y-direction. Here, a position higher by Δy12 than the height y1 of a lower inner wall surface B1 of the bore B of the gantry 2 in the y-direction is defined as the prespecified position y2. Δy12 may be of the order of 5 to 10 cm, for example. The table 4 has not reached y=y2 yet here. Therefore, the process goes back to Step S6.

Similarly thereafter, in the case that it is decided that the body part to be imaged is not detected at Step S7, and at the same time it is decided that the cradle 41 has not reached y=y2 at Step S8, a loop of Steps S6, S7, and S8 is repetitively executed.

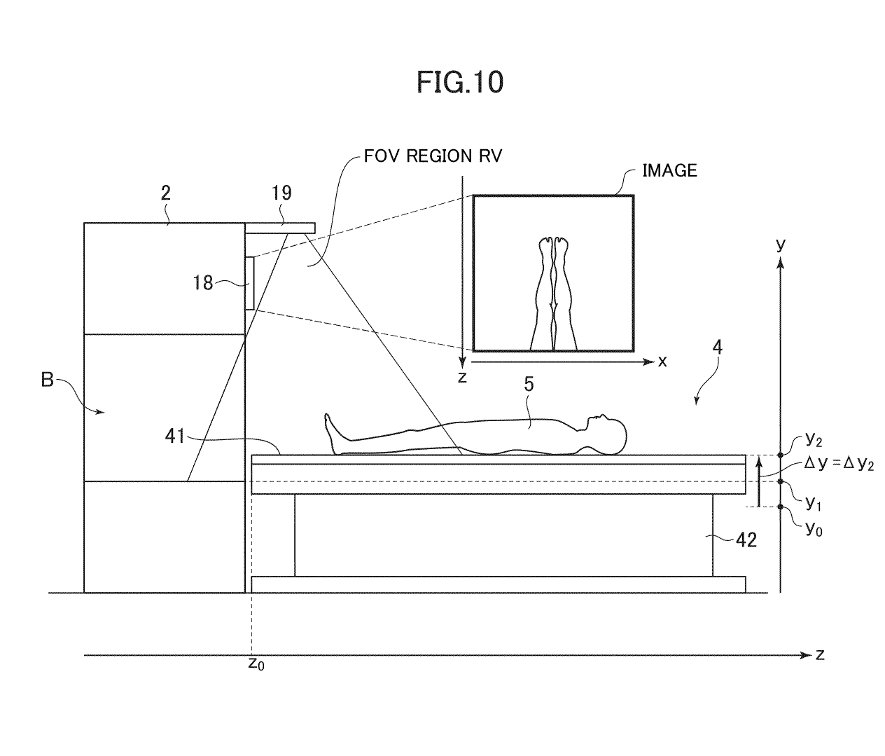

FIG. 10 is a diagram showing a condition in which the cradle 41 has reached y=y2.

While the cradle 41 is moving from y=y0 to y=y2, the image producing section 101 produces an image at Step S6 each time the position of the cradle 41 in the y-direction is changed. Therefore, while the cradle 41 is moving in the y-direction, the image displayed in the display section 18 is updated to the latest one. Moreover, the height-data generating section 103 generates height data at Step S6 each time the position of the cradle 41 in the y-direction is changed. Therefore, while the cradle 41 is moving in the y-direction, the height data is updated to the latest height data. Each time the height data is updated, the detecting section 104 executes the processing for detecting the chest part as the body part to be imaged of the subject 5 at Step S7 based on the latest height data.

While the cradle 41 is moving from y=y0 to y=y2, the body part to be imaged (chest part) of the subject falls outside the field-of-view region RV, so that it is decided that the body part to be imaged is not detected at Step S7. Once the cradle 41 has reached y=y2 as shown in FIG. 10, however, the cradle-position deciding section 105 decides that the cradle 41 has reached the prespecified position y=y2, so that the process goes to Step S9.

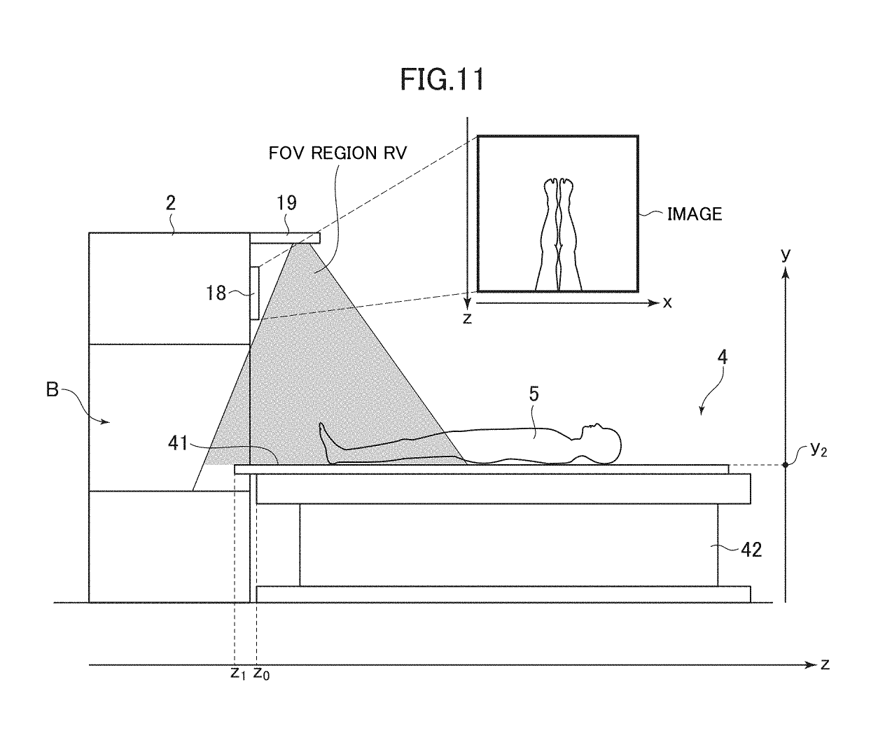

At Step S9, the cradle 41 stops moving in the y-direction, and starts moving in the z-direction. FIG. 11 is a diagram showing a condition when the cradle 41 has moved from z=z0 to z=z1.

At Step S10, the image producing section 101 produces an image of the subject 5 based on image data obtained from the sensor section 19. The display control section 102 controls the display section 18 so that the image produced by the image producing section 101 is displayed in the display section 18. In FIG. 11 is schematically shown the image at a time point when the cradle 41 has reached z=z1.

Moreover, the height-data generating section 103 generates height data at the time point when the cradle 41 has reached z=z1 based on distance data obtained from the sensor section 19.

At Step S11, the detecting section 104 executes the detection processing for detecting the body part to be imaged of the subject from among the height data generated at Step S10. In the case that the body part to be imaged is detected, the process goes to Step S12. On the other hand, in the case that the body part to be imaged is not detected, the process goes back to Step S10. In FIG. 11, the body part to be imaged (chest part) does not fall within the field-of-view region RV. Therefore, the body part to be imaged is not detected, and the process goes back to Step S10.

Similarly thereafter, a loop of Steps S10 and S11 is repetitively executed until it is decided that the body part to be imaged is detected at Step S11.

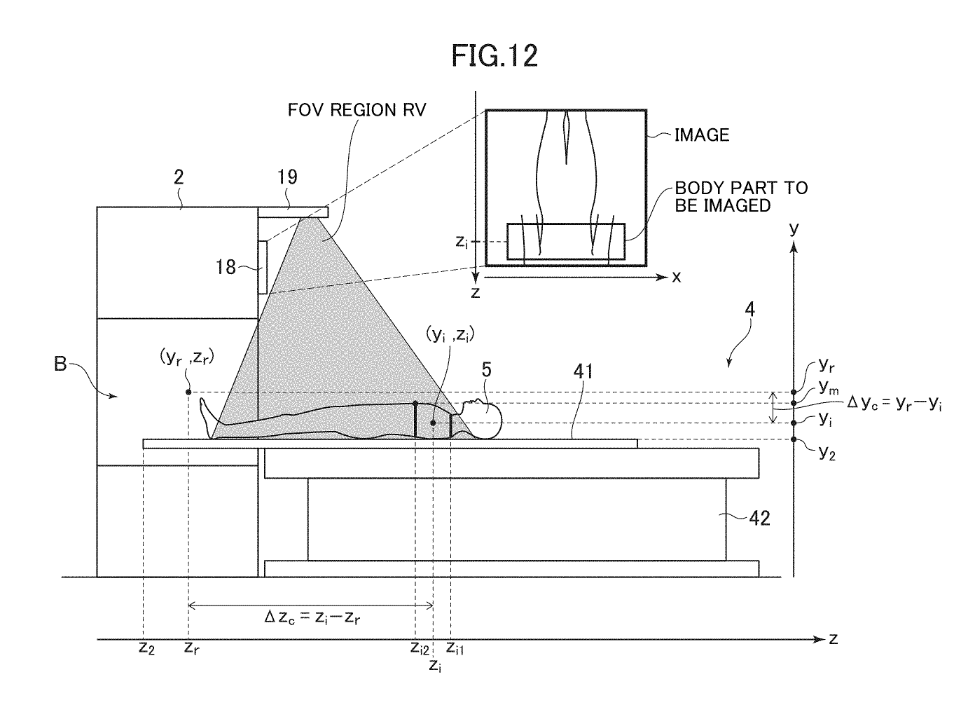

FIG. 12 is a diagram showing a condition in which the cradle 41 has reached z=z2.

While the cradle 41 is moving in the z-direction, the image producing section 101 produces an image at Step S10 each time the position of the cradle 41 in the z-direction is changed. Therefore, while the cradle 41 is moving in the z-direction, the image displayed in the display section 18 is updated to the latest one. Moreover, the height-data generating section 103 generates height data at Step S10 each time the position of the cradle 41 in the z-direction is changed. Therefore, while the cradle 41 is moving in the z-direction, the height data is updated to the latest height data. Each time the height data is updated, the detecting section 104 executes the processing for detecting the chest part as the body part to be imaged of the subject 5 at Step S11 based on the latest height data.

When the cradle 41 has reached z=z2, the chest part as the body part to be imaged of the subject 5 falls within the imaging field of view RV. Therefore, the detecting section 104 detects the chest part as the body part to be imaged of the subject 5 from among the height data. In FIG. 12, the position of the chest part is designated by (yi, zi). The position yi of the chest part in the y-direction is calculated as a mid-position between a maximum ym of the position in the y-direction on the body surface of the chest part of the subject, and the position y2 of the cradle 41 in the y-direction, that is, y=(ym−y2)/2. The position zi of the chest part in the z-direction is calculated as a mid-position in a range from zi1 to zi2 of the chest part of the subject in the z-direction, that is, z=(zi1−zi2)/2.

After detecting the chest part, the process goes to Step S12.

At Step S12, the calculating section 106 (see FIG. 6) calculates amounts Δyc and Δzc of movement of the cradle 41 required for positioning the chest part of the subject 5 at a prespecified position (yr, zr) in the bore B of the gantry 2. Δyc is the amount of movement of the cradle 41 in the y-direction required for positioning the chest part of the subject 5 at the prespecified position yr in the bore B of the gantry 2 in the y-direction. On the other hand, Δzc is the amount of movement of the cradle 41 in the z-direction required for positioning the chest part of the subject 5 at the prespecified position zr in the bore B of the gantry 2 in the z-direction. Since the position of the body part to be imaged in the y-direction is yi, the calculating section 106 calculates Δyc=yr−yi. Moreover, since the position of the body part to be imaged in the z-direction is zi, the calculating section 106 calculates Δzc=zi−zr. After calculating the amounts Δyc and Δzc of movement, the process goes to Step S13.

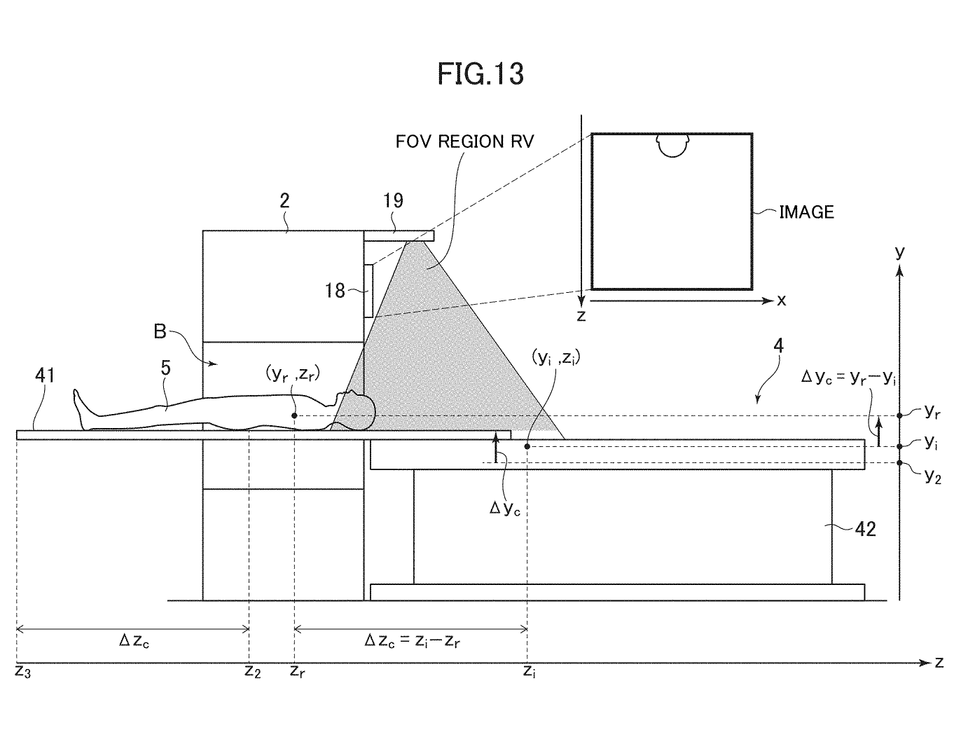

At Step S13, the amount-of-movement deciding section 107 (see FIG. 6) decides whether or not the cradle 41 has moved by Δyc in the y-direction and by Δzc in the z-direction. The decision at Step S13 is performed until it is decided that the cradle 41 has moved by Δyc and Δzc. FIG. 13 shows a condition in which the cradle 41 has moved by Δyc and Δzc. By the cradle 41 having moved by Δyc and Δzc, the body part to be imaged is positioned from (yi, zi) to (yr, zr). Once the cradle 41 has moved by Δyc and Δzc as shown in FIG. 13, the process goes to Step S14 and the cradle 41 stops. The process then goes to Step S15, where a scan on the subject 5 is performed and the flow is terminated.

According to the present embodiment, the field-of-view region of the sensor section 19 is defined so that the portion 4a of the table 4 on the side of the gantry 2 falls within the field-of-view region RV while the portion 4b of the table 4 on the side opposite to the gantry 2 falls outside the field-of-view region RV. Since the sensor section 19 can thus be installed at a position closer to the subject 5, accuracy of detection of the distance data can be improved. Since the height data can thus be obtained with high quality, accuracy of detection of the body part to be imaged can be improved.

Moreover, since a region to be detected by the imaging section in the sensor section 19 can be smaller by excluding the portion 4b of the table 4 on the side opposite to the gantry 2 from the field-of-view region RV, an image with higher resolution and reduced distortion can be displayed in the display section 18. Therefore, a high-quality image can be provided to the radiographer.

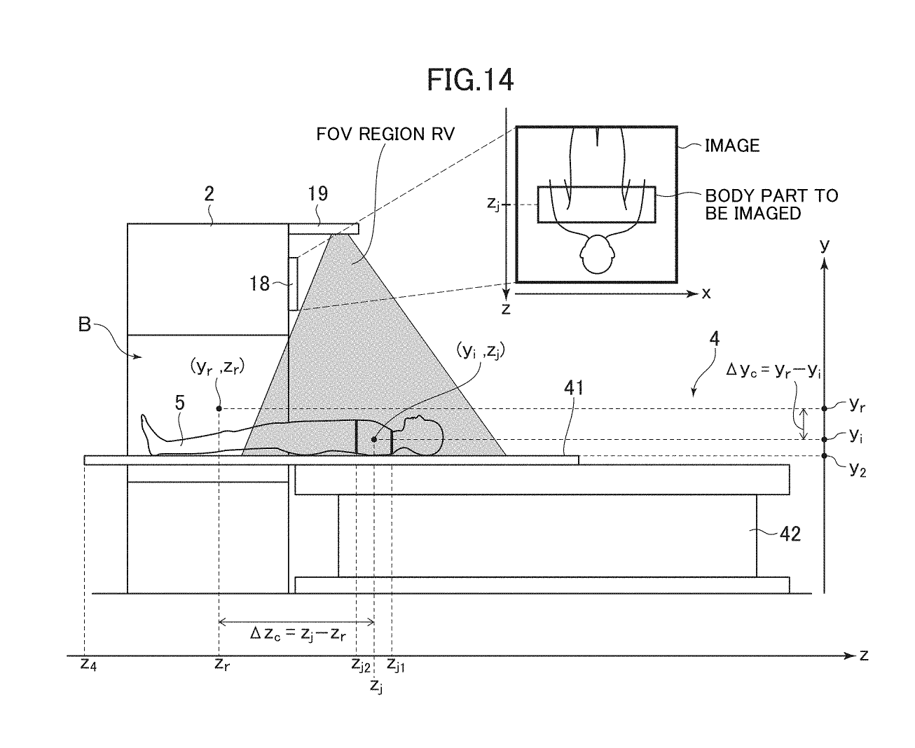

According to the present embodiment, the amounts Δyc and Δzc of movement of the cradle 41 required for carrying the body part to be imaged to a prespecified position are calculated when the body part to be imaged has reached z=zi (see FIG. 12). The amount Δz of movement, however, may be calculated when the body part to be imaged is moved to a position offset from z=zi insofar as the body part to be imaged falls within the field-of-view region RV. For example, as shown in FIG. 14, an amount Δzc (=zj−zr) of movement may be calculated when the body part to be imaged has reached a center position zj of the field-of-view region RV in the z-direction.

According to the present embodiment, a case is described in which once the body part to be imaged of the subject 5 has been detected, the cradle 41 is moved until the body part to be imaged is carried into the bore B. The cradle 41, however, may be stopped at a time point when the body part to be imaged is detected. By stopping the cradle 41 at the time point when the body part to be imaged is detected, the radiographer can confirm the posture of the subject 5 before the body part to be imaged is carried into the bore B. Thus, the radiographer can finely adjust the posture of the subject 5 before the body part to be imaged is carried into the bore B, and therefore, the body part to be imaged of the subject 5 can be carried into the bore B after finely adjusting the posture of the subject 5 to one suitable for imaging. Moreover, by viewing the display section 18, the radiographer can visually confirm which body part of the subject 5 is detected as the body part to be imaged before the body part to be imaged is carried into the bore B. Therefore, in the case that the detected body part to be imaged is not the chest part, the radiographer can reconfirm scan conditions before carrying the body part to be imaged of the subject 5 into the bore B. Furthermore, by stopping the cradle 41 at the time point when the body part to be imaged is detected, the radiographer can finely adjust the position of the cradle 41 before the body part to be imaged is carried into the bore B.

According to the present embodiment, a case is described in which templates corresponding to body parts to be imaged are prepared for use to detect a body part to be imaged from among the height data. However, a method of detecting a body part to be imaged is not limited to the method. For example, it may be contemplated to identify as a referential body part a body part (the shoulder part, for example) that has a characteristic shape and is easy to identify from among the height data, and detect the position of a body part to be imaged based on the position of the referential body part. In the case that the method involving identifying the position of the body part to be imaged based on the position of the referential body part is employed, it is preferable to, for each body part to be imaged, determine beforehand a distance between the referential body part and the body part to be imaged, and store in the storage section a table representing a correspondence between each body part to be imaged and the distance. Once the referential body part has entered the field-of-view region, the detecting section 104 can detect the referential body part from the image. Thus, the detecting section 104 can detect the position of the body part to be imaged by retrieving a distance between the referential body part and body part to be imaged from the correspondence table.

Moreover, according to the present embodiment, the sensor section 19 is provided for each pixel with an imaging section for acquiring image data and a light-receiving section for acquiring distance data. The sensor section 19, however, is not limited to that of the type provided with the imaging section and light-receiving section for each pixel, and may have a configuration comprising separate image sensor for acquiring image data and sensor for acquiring distance data (a depth sensor, for example).

Furthermore, according to the present embodiment, the sensor section 19 is configured to acquire image data and distance data; however, it is possible to configure it to acquire only distance data without acquiring image data.

While in the present embodiment, the present invention is described focusing upon a CT apparatus, it may be applied to any medical apparatus (for example, an MRI apparatus) different from the CT apparatus.

What is claimed is:

1. A medical apparatus comprising:
a gantry;
a table on which a subject to be examined is laid;
a sensor section for acquiring distance data for determining a distance between a body part to be imaged of said subject and said sensor section;
height-data generating means for generating height data containing data representing a height of the body part to be imaged of said subject based on said distance data; and
detecting means for detecting said body part to be imaged based on said height data, wherein
said sensor section has a field-of-view region representing a region in which it is possible to acquire said distance data, and
said field-of-view region is defined so that a portion of said table on a side of said gantry falls within said field-of-view region and a portion of said table on a side opposite to the side of said gantry falls outside said field-of-view region.

2. The medical apparatus as recited in claim 1, wherein:
said detecting means detects, based on information for identifying the body part to be imaged of said subject, the body part to be imaged identified by said information from among said height data.

3. The medical apparatus as recited in claim 1, wherein:
said height-data generating means updates said height data to latest height data while said table is moving, and
said detecting means detects said body part to be imaged from among the updated height data.

4. The medical apparatus as recited in any one of claims 1, comprising: calculating means for calculating, when said body part to be imaged is detected, an amount of movement of said cradle required for positioning said body part to be imaged at a prespecified position in a bore of said gantry.

5. The medical apparatus as recited in claim 4, wherein:
said cradle is movable in a first direction corresponding to a direction of a body axis of the subject and in a second direction corresponding to a vertical direction, and
said calculating means calculates as the amount of movement of said cradle a first amount of movement of said cradle in said first direction and a second amount of movement of said cradle in said second direction.

6. The medical apparatus as recited in any one of claims 1, wherein: said sensor section uses a light source emitting light onto said subject to acquire said distance data.

7. The medical apparatus as recited in claim 6, wherein:
said sensor section has a light-receiving section for receiving light emitted from said light source and reflected off said subject, and
said sensor section acquires said distance data based on reflected light received at said light-receiving section.

8. The medical apparatus as recited in claim 7, wherein: said sensor section has an imaging section for acquiring image data of said subject.

9. The medical apparatus as recited in claim 8, wherein: said imaging section is a CCD for acquiring color information.

10. The medical apparatus as recited in claim 8, wherein:
said sensor section has a number of pixels of n by m, and
each pixel comprises said light-receiving section and said imaging section.

11. The medical apparatus as recited in claim 1, wherein:
said sensor section has an image sensor for acquiring image data of said subject and a depth sensor for acquiring said distance data.

12. A computer readable storage medium storing a program applied to a medical apparatus comprising: a gantry; a table on which a subject to be examined is laid; and a sensor section for acquiring distance data for determining a distance between a body part to be imaged of said subject and said sensor section, wherein said sensor section has a field-of-view region representing a region in which it is possible to acquire said distance data, and said field-of-view region is defined so that a portion of said table on a side of said gantry falls within said field-of-view region and a portion of said table on a side opposite to the side of said gantry falls outside said field-of-view region, said program causing a computer to execute:
height-data generating processing of generating height data containing data representing a height of the body part to be imaged of said subject based on said distance data; and
detecting processing of detecting said body part to be imaged based on said height data.
